(19) United States
(12) Patent Application Publication
(10) Pub. No.: US 2025/0222248 A1
(43) Pub. Date: Jul. 10, 2025

(54) METHOD OF PURGING A BLOOD PUMP

(71) Applicant: ABIOMED, Inc., Danvers, MA (US)

(72) Inventors: Soumen Das, Danvers, MA (US); Vladimir Gilman, Danvers, MA (US); Alexander Ship, Danvers, MA (US); Scott C. Corbett, Danvers, MA (US); Kar Lee, Danvers, MA (US)

(73) Assignee: ABIOMED, Inc., Danvers, MA (US)

(21) Appl. No.: 19/014,872

(22) Filed: Jan. 9, 2025

Related U.S. Application Data
(63) Continuation of application No. 17/237,733, filed on Apr. 22, 2021, now abandoned.
(60) Provisional application No. 63/017,445, filed on Apr. 29, 2020.

(51) Int. Cl. A61M 60/829

(52) U.S. Cl. CPC A61M 60/829; A61M 2202/0085; A61M 2205/36

(57) ABSTRACT

A method of operating a blood pump that is purged with a purge solution that contains a pH controlling and buffering agent combined with aqueous dextrose and a reduced amount of heparin or no heparin. The blood pump has a gap between a shaft and housing through which the purge solution flows. In operation, the purge solution exits the housing and is discharged into the patient. The purge solution is therefore required to perform lubricating and heat transfer functions in the pump yet be biocompatible with the patient.

### CROSS-REFERENCE TO RELATED APPLICATION

This application is a continuation of U.S. application Ser. No. 17/237,733, filed Apr. 22, 2021, which claims the benefit of U.S. Provisional Application No. 63/017,445, filed Apr. 29, 2020, the disclosures of all of which are incorporated herein by reference in their entirety.

### TECHNICAL FIELD

This invention relates to a blood pump, in particular an intravascular blood pump, to support a blood flow in a patient's blood vessel and methods for purging such a pump in operation while inserted into a patient.

### BACKGROUND

Blood pumps of different types are known, such as axial blood pumps, centrifugal blood pumps, or mixed-type blood pumps, where the blood flow is caused by both axial and radial forces. One example of a blood pump is the Impella line of blood pumps (e.g., Impella 2.5®, Impella CP®, Impella 5.5®, etc.) which are products of Abiomed of Danvers, MA. Intravascular blood pumps are inserted into a patient's vessel such as the aorta by means of a catheter.

In some pump designs, a purge fluid is deployed to keep blood from entering the mechanism and to mitigate the effects of blood on the pump mechanisms, an anticoagulant such as heparin (typically the sodium salt of heparin). The heparin is thought to keep the blood from coagulating in the gap between pump components such as an impeller shaft and the housing. Heparin is a commonly used anticoagulant typically administered in controlled dosages.

In one example, the purge fluid is delivered by a purge cassette that enters a blood pump catheter through a filter assembly and internal purge lumen that carries the purge fluid through the catheter to a purge channel in the motor assembly. The flow of the purge fluid is regulated by an automated controller.

### BRIEF SUMMARY

Described herein is a method for purging a blood pump. According to the described method a blood pump that may include a motor section and a pump section is provided. At least a portion of the blood pump is inserted into a patient. The method also includes operating the blood pump to: i) provide a purge fluid to the motor section, where the purge fluid flows into gaps between a bearing in a motor housing of the motor section; and ii) cause an impeller in the pump section to rotate based on a rotation of a shaft in the motor section by a motor in the motor section. The purge fluid may include a pH controlling and buffering agent.

Previously, such purge fluids contained heparin. However, doctors often do not want heparin to be administered to the patient's blood via the purge fluid. For instance, administration of heparin during any sort of surgical procedure may be counterproductive as it prevents the coagulation of blood and, thus, healing or hemostasis. Also, the amount of heparin administered to the patient's blood along with the purge fluid is difficult to control for various reasons. In particular, the amount of heparin is often more than what is desired by the doctors, and the amount of heparin administered to the patient is difficult to precisely controlled. Accordingly, doctors would often prefer to supply heparin to the patient separate from the operation of the blood pump, if needed (and then only in the amount needed). Furthermore, some patients are heparin-intolerant because they are susceptible to heparin-induced thrombocytopenia (HIT). So, a heparin-containing purge is not at all suitable for these patients. Also, salts of heparin can cause unwanted wear on pump bearings that are made of metal. Accordingly, there is a need for an intravascular blood pump which can run, if desired, with a purge fluid that contains no or at least a reduced amount of heparin.

In one aspect, the purge fluid further includes aqueous dextrose. In another aspect the purge fluid also includes reduced amounts of heparin. An amount of heparin in the purge fluid is about zero to about 12.5 units per milliliter. In another aspect, the amount of heparin in the purge fluid is about zero to about 6.25 units per milliliter. In another aspect the amount of heparin in the purge fluid is about 1 units per milliliter to about 6.25 units per milliliter. In one aspect the pH controlling and buffering agent is one of sodium bicarbonate, citrate, lactate, gluconate, acetate and pyruvate. In the embodiment wherein the pH controlling and buffering agent is sodium bicarbonate, the amount of the pH controlling agent in the purge fluid is about 1.5 milliequivalents per liter (meq/l) to about 50 meq/l. The pH of the pH controlling and buffering agent is about 7.5 to about 9.1.

### BRIEF DESCRIPTION OF DRAWINGS

Hereinafter, the invention will be explained by way of example with reference to the accompanying drawings. The accompanying drawings are not intended to be drawn to scale. In the drawings, each identical or nearly identical component that is illustrated in various figures is represented by a like numeral. For purposes of clarity, not every component may be labelled in every drawing. In the drawings:

FIG. 1 illustrates the blood flow and the purge flow through the gap between the shaft and the housing in the pump;

FIG. 2 is a schematic representation of an intravascular blood pump inserted before the left ventricle, with its inflow cannula positioned in the left ventricle;

FIG. 3 is a schematic longitudinal cross-section of an exemplary prior art blood pump; and

FIG. 4 is an enlarged representation of a part of the blood pump of FIG. 3 according to a second embodiment.

### DETAILED DESCRIPTION

Blood pumps are deployed in patients that require critical and life-saving care. Consequently, it is important to remediate any aspect of the device that might adversely affect pump operation. Disclosed herein is the operation of blood pump in which the purge fluid contains sodium bicarbonate in addition to or in place of heparin.

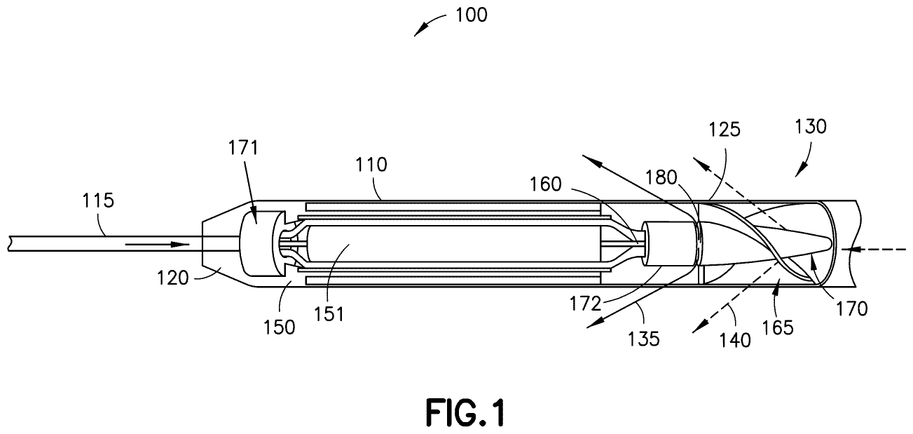

A blood pump of the aforementioned type is known, e.g., from EP 0 961 621 B1. With reference to FIG. 1, a pump 100 which possesses a drive section 110, a catheter 115 attached to the proximal end 120 of the drive section 110 (which is the end of the drive section closer to the doctor or “rear end” of the drive section) and having lines extending therethrough for the power supply to the drive section 110, and a pump section 130 fastened at the distal end 125 of the drive section. The drive section 110 comprises a motor housing 150 having an electric motor 151 disposed therein, with the motor shaft 160 of the electric motor distally protruding out of the drive section 110 and into the pump section 130. The pump section 130 in turn comprises a tubular pump housing 165 having an impeller 170 rotating therein which is seated on the end of the motor shaft 160 protruding out of the motor housing 150. The motor shaft 160 is mounted in the motor housing in two bearings 171, 172 which are maximally removed from each other in order to guarantee a true, exactly centered guidance of the impeller 170 within the pump housing 150. Different bearing types are used in different pump designs. As illustrated in FIG. 1, bearing 171 is a radial ball bearing and bearing 172 is an axial-radial sliding bearing. As illustrated in FIG. 1, blood 140 exits the outflow cage of the pump housing 165. Blood that would otherwise enter into the motor housing 150 is furthermore counteracted by a purge fluid 135 being passed through the motor housing and the impeller-side shaft seal bearing. Accordingly, the purge fluid passes through the gap of the impeller-side radial sliding bearing so as to prevent blood from entering into the housing. This is done at a purge fluid pressure that is higher than the pressure present in the blood.

As illustrated in FIG. 1, the purge fluid 135 fills the motor housing 150 of the pump to form a lubricating film in the bearings 171, 172 of the pump. As described in US Patent Publication No. 20150051436, the purge fluid 135 can form a lubricating film in a bearing gap 180 of the axial slide bearing of a pump. Purge fluids are described as being fed through a purge-fluid feed line and flowing through the radial bearing 171 located at the distal end of the motor housing 150 and then also flowing through the bearing gap 180 of the axial sliding bearing. The purge fluids fed in this manner are responsible for hemo-dilution and reduce blood retention time under the impeller 170.

To ensure that the purge fluid 135 reaches the distal radial bearing 172 at a pressure higher than the blood pressure present, there is provided, in at least one of the surfaces forming the bearing gap of the axial sliding bearing, a channel which penetrates the bearing gap 180 from radially outward to radially inward, so that the purge fluid can flow through this channel to the distal radial bearing. This channel need not necessarily lie in a bearing-gap surface, but can also be realized as a separate channel or as a bore. However, providing the channel in one of the bearing-gap surfaces has the advantage that the lubricating film in the bearing gap heats up less, because a part of the lubricating film is continually being replaced by purge fluid flowing in later. Preferably, the channel is located in the stationary bearing-gap surface in order to minimize the radial conveying capacity.

A general problem arises with the heparin that is typically mixed into the purge fluid. That is, despite the purge fluid flowing through the gap formed between the shaft and the opening of the housing, thereby pushing back the blood which tends to enter the housing through such gap, blood ingress into the gap cannot entirely be prevented. In particular, some blood or blood components may always enter at least into a distal section of such gap. Heparin helps to prevent coagulation of the blood in the gap or adhesion of blood to the surfaces and, thus, prevents blockage of shaft rotation.

EP 3 542 837 A1 describes a pump that limits the use of a purge fluid, at least intermittently, to mitigate the consequences of the administration of heparin to a patient through the blood pump purge fluid. To accomplish this, EP 3 542 837 A1 proposes using a material for at least one surface of the sliding bearing having a relatively high thermal conductivity for the gap surfaces. Examples of such materials include silicon carbide. The opposing surface can be made of a ceramic material with a lower thermal conductivity (e.g., alumina toughened zirconia). As described, the shaft is made of alumina toughened zirconia and the sleeve in which the shaft is journaled is made of silicon carbide. Using special materials for pump components is therefore one solution that limits or even eliminates the use of a heparin-containing purge fluid.

However, a more universal solution to the difficulties of using heparin in purge fluids for heart pumps continues to be sought.

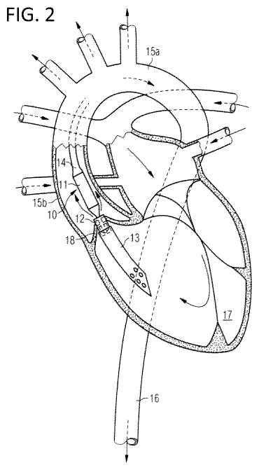

FIG. 2 represents the employment of a blood pump for supporting, in this particular example, the left ventricle. The blood pump comprises a catheter 14 and a pumping device 10 attached to the catheter 14. The pumping device 10 has a motor section 11 and a pump section 12 which are disposed coaxially one behind the other and result in a rod-shaped construction form. The pump section 12 has an extension in the form of a flexible suction hose 13, often referred to as “cannula.” An impeller is provided in the pump section 12 to cause blood flow from a blood flow inlet to a blood flow outlet, and rotation of the impeller is caused by an electric motor disposed in the motor section 11. The blood pump is placed such that it lies primarily in the ascending aorta 15b leading to the aortic arch 15a. The aortic valve 18 comes to lie, in the closed state, against the outer side of the pump section 12 or its suction hose 13 that lies substantially in the left ventricle 17. The blood pump with the suction hose 13 in front is advanced into the represented position by advancing the catheter 14, optionally employing a guide wire. In so doing, the suction hose 13 passes the aortic valve 18 retrograde, so the blood is sucked in through the suction hose 13 and pumped into the aorta 16.

The use of the blood pump is not restricted to the application represented in FIG. 2, which merely involves a typical example of application. Thus, the pump can also be inserted through other peripheral vessels, such as the subclavian artery. Alternatively, reverse applications for the right ventricle may be envisioned.

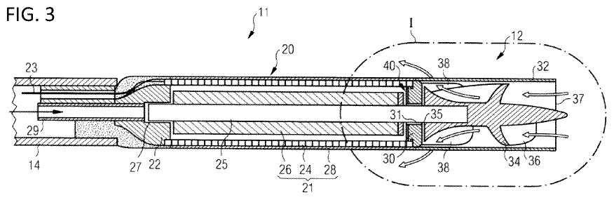

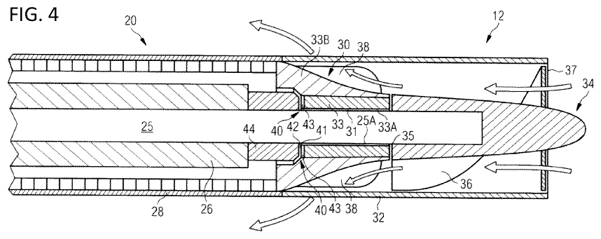

FIG. 3 shows an exemplary embodiment of the blood pump as described in US Patent Publication No. 2015/0051436 A1, which is likewise suitable for use in the context of the present invention, except that the encircled front end marked with “I” may be modified, such modification being shown in FIG. 4. Accordingly, the motor section 11 has an elongated housing 20 in which an electric motor 21 may be housed. A stator 24 of the electric motor 21 may have, in the usual way, numerous circumferentially distributed windings as well as a magnetic return path 28 in the longitudinal direction. The magnetic return path 28 may form an outer cylindrical sleeve of the elongate housing 20. The stator 24 may surround a rotor 26 connected to the motor shaft 25 and consisting of permanent magnets magnetized in the active direction. The motor shaft 25 may extend over the entire length of the motor housing 20 and protrude distally out of the latter through an opening 35. There, it carries an impeller 34 with pump vanes 36 projecting therefrom, which may rotate within a tubular pump housing 32 which may be firmly connected to the motor housing 20.

The proximal end of the motor housing 20 has the flexible catheter 14 sealingly attached thereto. Through the catheter 14, there may extend electrical cables 23 for power supply to and control of the electric motor 21. In addition, a purge fluid line 29 may extend through the catheter 14 and penetrate a proximal end wall 22 of the motor housing 20. Purge fluid may be fed through the purge fluid line 29 into the interior of the motor housing 20 and exit through the end wall 30 at the distal end of the motor housing 20. The purging pressure is chosen such that it is higher than the blood pressure present, in order to thereby prevent blood from penetrating into the motor housing, being between 300 and 1400 mmHg depending on the case of application.

As mentioned before, the same purged seal can be combined with a pump that is driven by a flexible drive shaft and a remote motor.

Upon a rotation of the impeller 34, blood is sucked in through the distal opening 37 of the pump housing 32 and conveyed backward within the pump housing 32 in the axial direction. Through radial outlet openings 38 in the pump housing 32, the blood flows out of the pump section 12 and further along the motor housing 20. This ensures that the heat produced in the motor is carried off. It is also possible to operate the pump section with the reverse conveying direction, with blood being sucked in along the motor housing 20 and exiting from the distal opening 37 of the pump housing 32.

The motor shaft 25 is mounted in radial bearings 27, 31 at the proximal end of the motor housing 20, on the one hand, and at the distal end of the motor housing 20, on the other hand. The radial bearings, in particular the radial bearing 31 in the opening 35 at the distal end of the motor housing, are configured as sliding bearings. Furthermore, the motor shaft 25 is also mounted axially in the motor housing 20, the axial bearing 40 likewise being configured as a sliding bearing. The axial sliding bearing 40 serves for taking up axial forces of the motor shaft 25 which act in the distal direction when the impeller 34 conveys blood from distal to proximal. Should the blood pump be used for conveying blood also or only in the reverse direction, a corresponding axial sliding bearing 40 may (also or only) be provided at the proximal end of the motor housing 20 in a corresponding manner.

FIG. 4 shows the portion marked with “I” in FIG. 3 in greater detail, yet structurally modified according to a preferred embodiment of the invention. There can be seen in particular the radial sliding bearing 31 and the axial sliding bearing 40. The bearing gap of the radial sliding bearing 31 is formed, on the one hand, by the circumferential surface 25A of the motor shaft 25 and, on the other hand, by the surface 33A of a through bore in a bushing or sleeve 33 of the motor housing's 20 end wall 30 defining an outer gap diameter of about 1 mm, but the outer gap diameter may also be larger than this. In one example, the bearing gap of the radial sliding bearing 31 has a gap width of 2 μm or less not only at the front end or impeller-side of the gap but over the entire length thereof. Preferably the gap width is between 1 μm and 2 μm. The length of the bearing gap may range from 1 mm to 2 mm, preferably from 1.3 mm to 1.7 mm, e.g., 1.5 mm. The surfaces forming the gap of the radial sliding bearing 31 have a surface roughness of 0.1 μm or less. These dimensions will vary with the type of pump and are presented by way of example and not by way of limitation.

The bearing gap of the axial sliding bearing 40 is formed, on the one hand, by the axially interior surface 41 of the end wall 30 and a surface 42 opposing it. This opposing surface 42 is part of a ceramic disk 44 which is seated on the motor shaft 25 distally of the rotor 26 and rotates with the rotor 26. A channel 43 in the bearing-gap surface 41 of the end wall 30 ensures that purge fluid can flow through between the bearing-gap surfaces 41 and 42 of the axial sliding bearing 40 to the radial sliding bearing 31 and exit from the motor housing 20 distally. The axial sliding bearing 40 represented in FIG. 3 is a normal sliding bearing. Unlike the representation, the axial gap of the axial sliding bearing 40 is very small, being a few μm.

Instead of the axial sliding bearing 40 and radial sliding bearing 31, there can also be realized a combined radial-axial sliding bearing 40 having a concave bearing shell in which a convex bearing surface runs. Such a variant is represented in FIG. 4 by a spherical sliding bearing 40. The bearing-gap surface 41 is of spherically concave design, and the opposing bearing-gap surface 42 is of corresponding spherically convex design. The channel 43 again lies in the stationary bearing-gap surface 41 of the end wall 30. Alternatively, the stationary bearing-gap surface 41 of the end wall 30 can be of convex configuration and the opposing bearing-gap surface 42 of concave configuration. The surfaces 42, 43 can also be conical instead of spherical. Preferably, a corresponding radial-axial sliding bearing is provided on both sides of the motor housing 20 in order not to permit any radial offset upon axial travel of the shaft 25. The advantage of a combined axial-radial sliding bearing lies in the higher loading capacity. However, a disadvantage is the greater frictional diameter.

During operation, the blood pump is attached to a purge-fluid source, and fluid passes into the motor housing through the purge-fluid line. The purge fluid then flows through the axial sliding bearing and further through the distal radial bearing. In the axial sliding bearing the purge fluid forms the lubricating film in the bearing gap. The pressure at which the purge fluid flows through the motor housing has an adverse effect, however, on the width of the bearing gap. Specifically, higher purge-fluid pressure requires a smaller bearing-gap width which results in a thinner lubricating film between the sliding surfaces.

The viscosity of the purge fluid is controlled by the concentration of dextrose in the purge fluid. Aqueous solutions of dextrose are widely administered to patients for a variety of reasons. The amount of dextrose in the aqueous solution is about 5% to about 50%. In one embodiment, the purge fluid contains 5% dextrose in water (i.e., 252 mmol/liter). The viscosity can be increased by including solutions with a higher concentration of dextrose in water (e.g., D20W, D40W, etc.). When a highly viscous purge fluid is used, the fluid film is maintained even at high pressures and the friction of the axial sliding bearing is accordingly independent of the purge-fluid pressure. In some embodiments, the axial sliding bearing can be configured as a simple sliding bearing, and does not have to be configured as a hydrodynamic sliding bearing, when a purge fluid having a viscosity at 37° C. that is about 1.2 mPas or higher. Therefore, when purge fluids that contain no or less heparin are considered, the viscosity of such purge fluids still needs to be considered.

The pump impeller does induce shear stress on the blood passing through the pump. Shear stress is induced predominantly in the gap between the impeller and the outer face of the ceramic bearing and between the impeller shaft and the inner face of the bearing (e.g., ceramic bearings, ball bearings, etc.). Due to the shear stresses to which the blood is subjected, blood proteins denature and polymerize as the blood passes through the pump. The deposition of the denatured and agglomerated protein causes activation of the clotting cascade, which, in turn, causes the build-up of bio-deposits on the pump mechanisms (e.g., the impeller, the outflow cage, etc.). Small gaps between components (i.e., purge gaps) are particularly vulnerable to blockage by bio-deposits. The bio-deposit build-up will cause the motor current needed to operate the pump to increase. The increased motor current or bio-deposits can degrade pump performance or even cause a pump stop.

As noted above, to mitigate the adverse effects of shear on the blood that flows through the pump, the purge fluids used in purged blood pumps typically include the anticoagulant heparin (e.g., 50 units/ml) in 5%-Dextrose (D5W). The dextrose concentration determines the viscosity of the purge fluid and hence affects the purge flow rate. Purge fluids with lower dextrose concentrations are less viscous and flow more quickly with less pressure through the purge system. Purge fluids with higher dextrose concentrations (more viscous) result in a lower purge flow rate and require a greater purge pressure. A reduction in dextrose concentration from 20% to 5% results in an approximately 30% to 40% increase in purge flow rates.

Purge flow rates are typically in the range of about 2 mL/hour to about 30 mL/hour. This results in a purge pressure of about 1100 mmHg to about 300 mmHg. Typical purge flows for the blood pumps described herein, e.g., Impella CP, Impella 2.5, Impella 5.0/LD, and RP, are about 5 mL/hour to about 20 mL/hour. These pumps all have a ball-bearing rotor/stator system with similar tolerances leading to similar purge operation ranges. Typical purge flows for the Impella 5.5 are about 2 to about 10 mL/hour. This lower flow rate results from the deployment of a ceramic bearing rotor/stator system designed with a reduced purge gap (radial) to reduce or eliminate the amount of heparin delivered to the patient. For surgical patients, surgeons prefer not to administer heparin in the first few days after surgery. For these patients, then, purge fluids that contain no heparin are preferred.

Consistent purge flow is used to keep two important regions clear of debris: 1) the gap between the rotor shaft and sleeve bearing; and 2) the gap between the sleeve bearing and the impeller. Due to diffusion and flow co-mixing, some blood components may potentially reach these gaps. Heparin in the purge solution enhances protection against ingress, adsorption, deposition, and coagulation of blood components. It also improves the working life of the bearings, for at least the reasons stated below.

Specifically, continuous and dynamic physical adsorption (physisorption) of heparin onto the surfaces around the purge path reduces adsorption of blood components and, thus, prevents bio-deposition of blood debris on the bearings and other pump components. Also, heparin partially neutralizes the slightly acidic D5W solution, which helps to maintain the physiological pH in the aforementioned gaps and, therefore, reduces the risk of blood protein denaturing. Locally elevated concentration of heparin, both under the impeller and inside the sleeve bearing gap, may also reduce the risk of blood coagulation in these areas. Addition of heparin increases the electric conductivity of the purge fluid and therefore reduces the negative impact of electrostatic discharge on the bearing working life.

Therefore, heparin is provided in the purge fluid to prevent the formation of shear-induced bio-material or bio-deposits, and the resulting undesirable deposition/accumulation of biological material in the pump, such as between the impeller shaft and the inner race of the bearings at high shear areas. However, as noted above, there are challenges associated with adding heparin to the purge fluid. Specifically, heparin: a) makes systematic anticoagulant management complex (i.e., there is a need to consider the heparin dose that the patient is receiving via the purge fluid); b) heparin, as an anticoagulant, increases a patient's propensity to bleed; c) heparin makes it more difficult to control bleeding in post-operative patients, especially when surgical devices are used on such patients; and d) heparin cannot be used for heparin-induced thrombocytopenia (HIT) patients. Also, heparin may also be administered systemically to some patients, making it difficult to regulate the administration of two source of heparin

However, a purge fluid/purge fluid additive that can mitigate problems in pump performance caused by the pump operation is still needed. It has been observed that the denatured proteins become prone to agglomeration as protein unfolding exposes hydrophobic regions of a protein. This causes unwanted bio-deposition. Absent denaturing and agglomeration, the hydrophobic segments are shielded, and protein molecules are repulsed due to the electrostatically charged groups of the protein.

Soluble calcium ions are known to mediate coagulation. The serum albumin in blood controls the calcium ions. At higher pH values, the albumin more strongly retains the calcium ions. This mechanism reduces the effective concentration of calcium available for coagulation. Therefore, providing an additive to the purge fluid that elevates the pH of the purge fluid will reduce the amount of calcium that will support coagulation in the high stress areas. Described herein are high pH buffers for use in a purge fluid that will elevate blood pH and therefore mitigate clotting.

Contemplated herein are pH-controlling and buffering agents that are added to the purge fluid that avoid the problems of heparin but meet the other objectives of the purge fluid (bio deposit mitigation; bearing wear reduction; higher pressure than blood pressure, etc.). One example of a suitable pH-controlling and buffering agent is sodium bicarbonate. However, pH-controlling and buffering agents other than sodium bicarbonate are also contemplated. Those pH controlling and buffering agents, include, for example, salts of small organic acids, such as citrate, lactate, gluconate, acetate, pyruvate, etc. In one example, the pH of sodium bicarbonate is about 7.4 to about 9.1. In one example, the pH of the purge fluid with bicarbonate is about 8.4. Other ranges, including but not limited to about 7.5 to about 9.1, 7.6 to about 9.1, 7.7 to about 9.1, 7.8 to about 9.1, 7.9 to about 9.1, 8.0 to about 9.1, 8.1 to about 9.1, 8.2 to about 9.1, 8.3 to about 9.1, 8.4 to about 9.1, 8.5 to about 9.1, 8.6 to about 9.1, 8.7 to about 9.1, 8.8 to about 9.1, 8.9 to about 9.1 and 9.0 to about 9.1 are contemplated. The pH of blood is about 7.3 to about 7.4. Adding sodium bicarbonate to the purge fluid will elevate the pH of the blood that comes into contact with the purge fluid. The elevated pH will reduce bio deposits that result from blood coagulation caused by the high shear pump environment. Due to this effect, the presence of sodium bicarbonate, even if coagulation proceeds towards formation of individual fibrin molecules, reduces the formation of insoluble bio deposits.

In one embodiment, adding a solution containing bicarbonate mixed with a dextrose solution such as dextrose 5% in water (D5W), dextrose 20% in water (D20W) dextrose 40% in water (D40W), etc. to blood increases the local pH of the blood at the gap (higher shear area) and prevents the agglomeration of the protein by increasing the electrostatic charge of the serum protein, and therefore reduces formation of bio-deposition. The amount of bicarbonate in the solution of bicarbonate mixed with the dextrose solution is about 1.5 milliequivalents per liter (mEq/L) to about 50 mEq/L. Other pH-controlling and buffering agents other than sodium bicarbonate are contemplated. Those pH controlling and buffering agents, include, for example, salts of small organic acids, such as citrate, lactate, gluconate, acetate, pyruvate, etc. The concentration of such other pH-controlling and buffering agents in the solution with aqueous dextrose is selected to provide a solution with a pH within the range prescribed above. The concentrations of these pH-controlling and buffering agents are selected so that their concentration in the solution do not significantly exceed the natural physiological limits of these buffering agents in blood (to the extent that such organic acids are present in the blood). Such concentration can easily be determined by one skilled in the art.

The purge solutions contemplate herein cause less bearing wear on the blood pumps than purge solutions that contain reduced heparin but do not contain the pH controlling and buffering agents described herein. It would be expected that bearing wear would increase with decreased amounts of heparin. Although applicants do not wish to be held to a particular theory, applicant submits that solutions with higher concentrations of heparin have a higher conductivity. Conductive purge solutions provide better charge dissipation, thereby reducing charge buildup on the metal bearings of the pump. Therefore, purge solutions with higher conductivity reduce the wear on the metal bearings. When the amount of heparin in the purge solution is reduced, the conductivity of the purge solution is also reduced. However, surprisingly, when the pH controlling and buffering agent is added to a purge fluid in addition to reduced amount or no amounts of heparin are in the purge fluid, the bearing wear does not increase because the pH controlling and buffering agent also increases the conductivity of the purge fluid. The purge solutions described herein also maintain the patency of the purge line that delivers the purge solution to the pump.

In some embodiments, the purge fluid solution may contain a reduced amount of heparin along with the pH controlling and buffering agents described above. Reduced concentrations of about 12.5 units/ml of heparin or less are contemplated. Reduced concentrations of about 6.25 units/ml or less are also contemplated. Reduced concentrations in the range of about 1 unit per ml to about 6.25 units/ml are also contemplated. Reducing the amount of heparin in the purge solution increases the amount of bearing wear, but the pH controlling and buffering agents described herein in the purge solution reduces the amount of bearing wear without the complications the ensue from the use of heparin in purge solutions that are described elsewhere herein.

Surprisingly, applicant has determined that the pH controlling and buffering agents described herein, when added to a dextrose-containing purge fluid alone or in combination with a reduced amount of heparin, mitigate the problem of bio-deposits on pump components that would otherwise result from the blood being subjected to the high shear of the pump impeller. Heparin is not required to be in the purge solution to mitigate the formation of the bio-deposits and to avoid pump stop.

Durability testing indicated no reduction of pump durability when using 25 units/mL in the purge solution along with a dextrose solution (e.g., 5% to 20% dextrose). A review of clinical data when 25 units/mL was used is in process was undertaken, but the initial findings did not indicate a difference in performance compared with 50 units/mL. In one example the units are mole equivalents in the solution.

If a patient is intolerant to heparin, due to heparin-induced thrombocytopenia (HIT), but there is still a need to add an anticoagulant to the purge solution of pH controlling and buffering agent combined with aqueous dextrose, a direct thrombin inhibitor (DTI) can be added to the solution. If a DTI is added to the purge solution, the concentration of the DTI in the purge solution should be a dose equivalent of about 0.01 mg/kg/hr. to about 0.012 mg/kg/hr. The dose equivalent is selected to provide a partial thromboplastin test (PTT) time of about 40-50 seconds. Examples of suitable DTIs include, but are not limited to argatroban or bivalirudin. The concentration of the DTI in the purge solution is about 20 mg/500 ml to about 60 mg/500 ml. When the DTI in the purge fluid is bivalirudin, for example, the concentration is about 20 mg/500 ml in a dextrose solution (e.g., D5W; D10W). When the DTI in the purge fluid is argatroban, for example, the concentration is about 30-60 mg/500 ml in a dextrose solution (e.g., D5W; D10W). When a DTI is added to the purge solution, it is added in place of heparin and not in addition to heparin.

Because bicarbonate preserves the purge line patency, when bicarbonate is present in the system, the purge solution mitigates the effect of kinks in the purge system, in addition to damage to purge elements or the build-up of bio-deposits in the pump.

One advantage of the purge solution described herein is that the pH controlling and buffering agent described herein is readily miscible in aqueous dextrose. Furthermore, when stored, the pH controlling and buffering agent remains mixed with the aqueous dextrose even when stored for a significant period of time. Heparin, by contrast, requires more aggressive mixing with the aqueous dextrose and will phase separate from the aqueous dextrose when stored for a significant period of time. However, when heparin is added to the purge solution containing the pH controlling and buffering agent in combination with the aqueous dextrose, the degree of ionization and net electronic charge of the heparin molecules increase. As a result, the heparin is more easily mixed with the purge solution and remains more evenly distributed in the bag. As a consequence, the current practice, which requires periodic equilibration of bag contents by manual squeezing the bags, can be eased or even eliminated.

Described herein are systems and methods that use sodium bicarbonate as an alternative to heparin in a purge fluid used to maintain the patency of a purge system for a blood pump. The sodium bicarbonate is deployed in the purge system and is not used for systemic coagulation of the patient in which the pump is deployed. Systemic anticoagulation using heparin, bivalirudin, or argatroban is used to prevent thromboembolic events, even when sodium bicarbonate is used in the purge.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

While particular embodiments of this technology have been described, it will be evident to those skilled in the art that the present technology may be embodied in other specific forms without departing from the essential characteristics thereof. The present embodiments and examples are therefore to be considered in all respects as illustrative and not restrictive. It will further be understood that any reference herein to subject matter known in the field does not, unless the contrary indication appears, constitute an admission that such subject matter is commonly known by those skilled in the art to which the present technology relates.

1.-28. (canceled)

29. Use of a purge fluid comprising a pH controlling and buffering agent for purging a blood pump:
wherein the blood pump comprises a motor section and a pump section,
wherein at least a portion of the blood pump is configured to be inserted into a patient and operated,
wherein, when the blood pump is operated, i) the purge fluid is provided to the motor section, wherein the purge fluid flows into a gap between a bearing in a motor housing of the motor section and a shaft in the motor section, and ii) the blood pump causes an impeller in the pump section to rotate based on a rotation of the shaft in the motor section by a motor in the motor section, and
wherein the pH controlling and buffering agent is configured to increase pH of blood and reduce blood protein denaturation in the gap.

30. The use of claim 29, wherein the purge fluid further comprises aqueous dextrose.

31. The use of claim 30, wherein the purge fluid further comprises heparin.

32. The use of claim 29, wherein the pH controlling and buffering agent is sodium bicarbonate, sodium citrate, sodium lactate, sodium gluconate, sodium acetate, or sodium pyruvate.

33. The use of claim 29, wherein the pH controlling and buffering agent is sodium bicarbonate.

34. The use of claim 33, wherein an amount of the pH controlling and buffering agent in the purge fluid is about 1.5 milliequivalents per liter (mEq/L) to about 50 mEq/L.

35. The use of claim 31, wherein an amount of heparin in the purge fluid is zero to about 12.5 units per milliliter.

36. The use of claim 31, wherein an amount of heparin in the purge fluid is zero to about 6.25 units per milliliter.

37. The use of claim 31, wherein an amount of heparin in the purge fluid is about 1 unit per milliliter to about 6.25 units per milliliter.

38. The use of claim 32, wherein the purge fluid further comprises a direct thrombin inhibitor.

39. The use of claim 38, wherein the direct thrombin inhibitor is argatroban, or bivalirudin, or mixtures thereof.

40. The use of claim 38, wherein an amount of the direct thrombin inhibitor in the purge fluid is about 20 mg/500 ml to about 60 mg/500 ml.

41. The use of claim 29, wherein pH of the pH controlling and buffering agent is about 7.4 to about 9.1.

42. The use of claim 29, wherein the pH controlling and buffering agent is not used for systemic anticoagulation of the patient into which at least a portion of the blood pump is inserted.
